(19) United States
(12) Patent Application Publication
(10) Pub. No.: US 2024/0236598 A1
(43) Pub. Date: Jul. 11, 2024

(54) SOUND MONITORING SYSTEM

(71) Applicant: OTOASSET INC, Taoyuan City (TW)

(72) Inventor: KANG-YOU PENG, Taoyuan City (TW)

(21) Appl. No.: 18/228,705

(22) Filed: Aug. 1, 2023

(30) Foreign Application Priority Data
Aug. 1, 2022 (TW) 111128777

(51) Int. Cl. H04S 7/00; G10L 15/08

(52) U.S. Cl. CPC H04S 7/301; G10L 15/083; H04S 7/40; H04S 2400/15

(57) ABSTRACT

A sound monitoring system is applied in a monitoring environment. The sound monitoring system includes a sound sensing module and a sound wave feature analysis module. The sound monitoring system receives a sound wave generated in the monitoring environment within a period of time, and the sound wave is converted into a sound wave feature data set through a conversion process, and the conversion process removes or reduces semantic components of the sound wave and the remained semantic components are unrecognizable. The sound wave feature analysis module, in communication with the sound sensing module, receives the sound wave feature data set and performs feature analysis and determination according to the sound wave feature data set, and then sends a control signal according to the feature analysis and judgment.

### FIELD OF THE INVENTION

The present disclosure relates to a sound monitoring system, and particularly to a sound monitoring system applied in a monitoring environment.

### BACKGROUND OF THE INVENTION

It is needed to monitor status of persons stay in hospital wards or long-term care institutions. However, there is insoluble conflict between privacy protection and information collection. For example, capturing surveillance images with a video camera is not allowed under rules of most countries. Further, monitoring with non-contact millimeter-wave radar will expose the human bodies to electromagnetic waves the whole day.

### SUMMARY OF THE INVENTION

The present disclosure relates to a sound monitoring system applied in a monitoring environment, including: a sound sensing module, receiving a sound wave generated in the monitoring environment throughout a period of time, wherein the sound wave is transformed into a sound wave feature data set through a transformation process and the transformation process removes or reduces semantic components of the sound wave to become unrecognizable; and a sound wave feature analysis module, in communication with the sound sensing module, receiving the sound wave feature data set and making feature analysis determination according to the sound wave feature data set, and then sending a control signal according to the feature analysis determination.

In an embodiment, the sound sensing module includes: a microphone continuously converting the sound wave generated in the monitoring environment into an electric wave signal in real time; and a feature value sampling device, electrically connected to the microphone and the sound wave feature analysis module, performing a feature value sampling procedure on the electric wave signal at every sampling period to obtain one or more feature values, and transmitting the one or more feature values to the sound wave feature analysis module.

In an embodiment, the sound wave feature analysis module includes: a memory device, electrically connected to the feature value sampling device, recording the feature values as a data file, wherein the data file includes feature value data corresponding to sampling time points; and a data processing unit, electrically connected to the memory device, making the feature analysis determination based on the data file in real time, and sending the control signal to an external system according to a determination result of the feature analysis determination.

In an embodiment, the feature value sampling device includes a signal amplifier and an analog-to-digital converter. The signal amplifier amplifies an amplitude of the electric wave signal. The analog-to-digital converter performs the feature value sampling procedure on the amplified electric wave signal at every sampling period. The feature value is obtained by transforming the amplitude of the amplified electric wave signal into an amplitude digital value. After obtaining one or more amplitude digital values, the one or more amplitude digital values are transmitted to the sound wave feature analysis module. The sound wave feature analysis module records and temporarily stores the one or more amplitude digital values into the memory device as a volume data file. The volume data file includes amplitude digital value data corresponding to the sampling time points.

In an embodiment, each of the amplitude digital values represents a volume at a corresponding sampling time point. The data processing unit makes the feature analysis determination based on the volume data file by using a time-varying amplitude digital value sequence recorded in the volume data file. The feature analysis determination includes steps of: determining that a monitored person has poor or no vital signs when the amplitude digital values falls in a preset region for a specific time period, and automatically sending the control signal to the external system. The external system is associated with a designated phone number or a user ID of instant messaging software, and the control signal is a warning signal calling a caregiver to give emergent treatment.

In an embodiment, the data processing unit compares a time-varying amplitude digital value sequence recorded in the volume data file and a preset value sequence; determines that a monitored person has weak or no vital signs when the data processing unit detects that the amplitude digital value sequence changes from a combination of a first waveform and a second waveform to just the second waveform which lasts for a specific time period; and automatically sends the control signal to the external system. The external system is associated with a designated phone number or a user ID of instant messaging software, and the control signal is a warning signal calling a caregiver to give emergent treatment.

In an embodiment, the feature value sampling device includes a conversion module and a fast Fourier transform module. The conversion module receives and converts the electric wave signal to have a lower frequency. The fast Fourier transform module using fast Fourier transform to perform the feature value sampling procedure at every sampling period, wherein a plurality of frequency spectrum values obtained at every sampling period form an array. The array is transmitted to the sound wave feature analysis module. The sound wave feature analysis module records and temporarily stores the frequency spectrum values into the memory device as a frequency spectrum data file. The frequency spectrum data file includes spectrum value data arrays corresponding to the sampling time points.

In an embodiment, the data processing unit compares the frequency spectrum data array of a specific time period recorded in the frequency spectrum data file and a preset value array; determines that a monitored person has weak or no vital signs when the data processing unit detects that the frequency spectrum data array changes from a combination of a first distribution and a second distribution into just the second distribution which lasts for a specific time period; and automatically sends the control signal to the external system. The external system is associated with a designated phone number or a user ID of instant messaging software, and the control signal is a warning signal calling a caregiver to give emergent treatment.

In an embodiment, a distribution state of the frequency spectrum data array is used to distinguish different sound types representing different respiratory abnormalities. The data processing unit determines that a monitored person has one respiratory abnormality when the data processing unit detects that a distribution corresponding to a combination of a background noise and the sound type representing the one respiratory abnormality conforms to a preset frequency spectrum data array.

In an embodiment, the sound sensing module further includes a mixer, wherein before the electric wave signal generated by the sound sensing module according to the sound wave is transmitted to the sound wave feature analysis module, the mixer introduces random noises, lying in a voice frequency band, into the electric wave signal to remove or reduce the semantic components of the sound wave to become unrecognizable.

In an embodiment, the sound sensing module includes: a microphone continuously converting the sound wave generated in the monitoring environment into an electric wave signal in real time; a voice detection module, electrically connected to the microphone, detecting a voice-active period in the electric wave signal; a voice labeling module, electrically connected to the voice detection module, labeling the voice-active period in the electric wave signal; a voice processing module electrically connected to the voice labeling module, wherein after the voice processing module receives the electric wave signal with labeling, the voice processing module generates an inverted waveform with an amplitude close to a waveform of the electric wave signal defined by the labeling, mixes both waveforms to remove or reduce the semantic components of the sound wave to become unrecognizable, and sends the electric wave signal with the semantic components removed; and a feature value sampling device, electrically connected to the voice processing module and the sound wave feature analysis module, performing a feature value sampling procedure on the electric wave signal with the semantic components removed at every sampling period to obtain one or more feature values, and transmitting the one or more feature values to the sound wave feature analysis module.

In an embodiment, the voice-active period is labeled by inserting specific signs at a start point and an end point of the voice-active period, or directly mixing a masker signal over the voice-active period to definitely indicate the voice-active period.

### BRIEF DESCRIPTION OF THE DRAWINGS

The advantages of the present disclosure will become more readily apparent to those ordinarily skilled in the art after reviewing the following detailed description and accompanying drawings, in which:

FIG. 1 is a functional block diagram of a sound monitoring system according to the present disclosure.

FIG. 2A is a functional block diagram of an embodiment of a feature value sampling device according to the present disclosure.

FIG. 2B is a schematic diagram showing the format of the volume data file according to the present disclosure.

FIG. 3 is a flowchart showing steps of an embodiment of the feature analysis determination according to the present disclosure.

FIG. 4A is a functional lock diagram of another embodiment of a feature value sampling device according to the present disclosure.

FIG. 4B is a schematic diagram showing another format of the volume data file according to the present disclosure.

FIG. 5 is a functional block diagram of another embodiment of a sound monitoring system according to the present disclosure.

FIG. 6 is a functional block diagram of a further embodiment of a sound monitoring system according to the present disclosure.

### DETAILED DESCRIPTION OF PREFERRED EMBODIMENTS

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

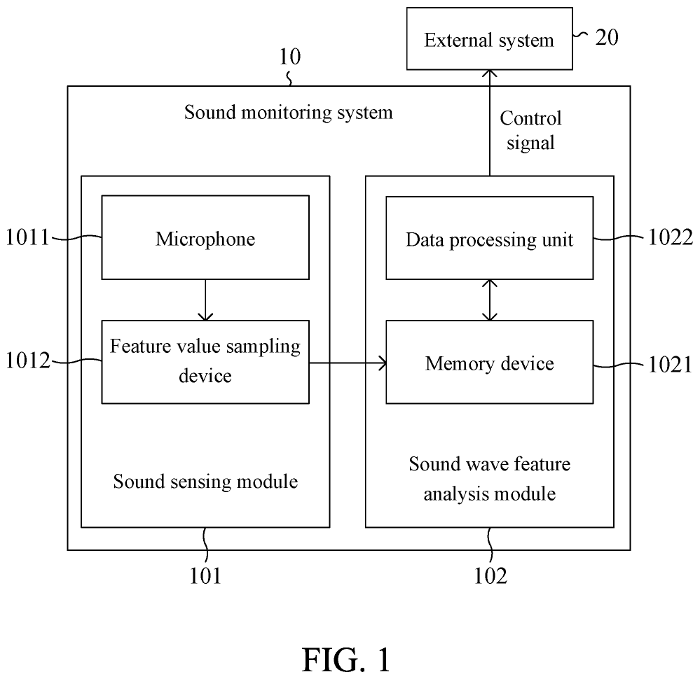

To improve the prior arts, the disclosure provides a sound monitoring system applied in a monitoring environment, and its functional block diagram is shown in FIG. 1. For example, the monitoring environment could be a bedroom in a nursing home where an elder lives and sleeps. The sound monitoring system 10 of the present disclosure basically includes a sound sensing module 101 and a sound wave feature analysis module 102. The sound sensing module 101 is configured to receive a sound wave generated in the monitoring environment throughout a period of time. The sound wave is transformed into a sound wave feature data set through a transformation process. The transformation process is characterized in that preselected features of the sound wave are remained while semantic components of the sound wave is removed or reduced to become unrecognizable. Thus, by analyzing the preselected features, indicators relating to the health status of the monitored person can be obtained. Because the sound wave feature data are obtained after removing the semantic components, the monitored person will not consider that his/her privacy has been violated.

For example, the sound sensing module 101 disposed in the monitoring environment includes a microphone 1011 which can continuously convert the sound wave generated in the bedroom into an electric wave signal in real time. Subsequently, a feature value sampling device 1012 performs a feature value sampling procedure on the electric wave signal at every sampling period to obtain and transmit one or more feature values to the sound wave feature analysis module 102. The sound wave feature analysis module 102 records and temporarily stores the feature value(s) into a memory device 1021 as a data file (could be considered as the sound wave feature data set described above). The data file mainly includes the feature value data corresponding to the sampling time points. A data processing unit 1022 of the sound wave feature analysis module 102 can make feature analysis determination based on the data file, and further sends a control signal to an external system 20 according to the determination result.

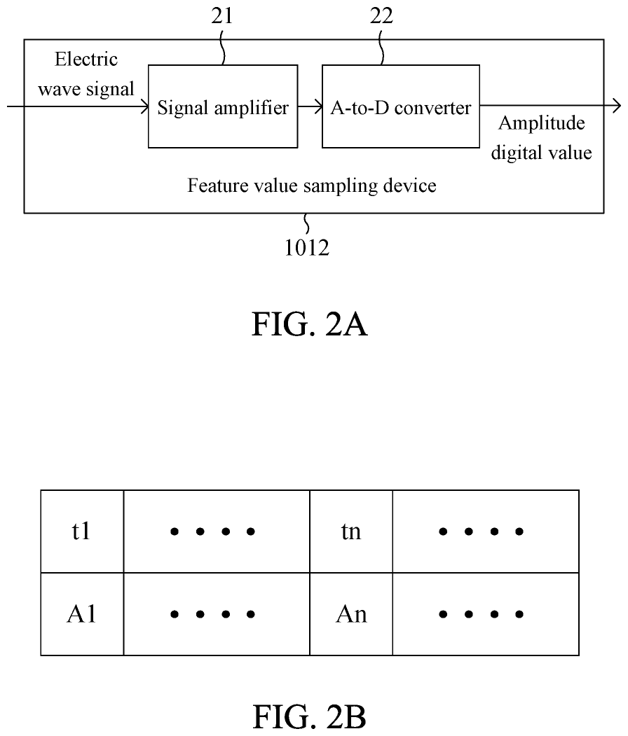

The feature value sampling device 1012 is exemplified herein. The feature value sampling device 1012, as shown in FIG. 2A, is a combination of a signal amplifier 21 and an analog-to-digital converter 22. The signal amplifier 21 is configured to amplify (increase) the amplitude of the electric wave signal, and the analog-to-digital converter 22 is configured to perform the feature value sampling procedure on the amplified electric wave signal at every sampling period. The feature value in the embodiment is obtained by transforming the amplitude of the amplified electric wave signal into an amplitude digital value. The one or more feature values (i.e. the amplitude digital value(s)) are then transmitted to the sound wave feature analysis module 102. The sound wave feature analysis module 102 records and temporarily stores the amplitude digital value(s) into the memory device 1021 as a volume data file. The volume data file could include, as shown in FIG. 2B, the amplitude digital value data A1 . . . An corresponding to the sampling time points t1 . . . tn.

The amplitude digital value represents the volume (i.e. decibel level) at the time point. The determination can be made according to the sequence of the time-varying amplitude digital values (or called a decibel spectrum) recorded in the volume data file. Although the volume data file keeps the amplitude feature of the sound wave, it only records the amplitude values of the sound wave, and the semantic components of the sound wave have been removed or reduced to become unrecognizable. Now, the data processing unit 1022 of the sound wave feature analysis module 102 can make the feature analysis determination based on the volume data file in real time to obtain the related indicators to determine the health status of the monitored person. Certainly, the analog-to-digital converter 22 could be replaced by an integrator to compute the integral of the signal intensity over a sampling period to obtain the representative signal intensity value corresponding to the sampling period.

After certain observation and statistical calculation, it is found that most sound sources can be classified. Generally speaking, there are three types of sound sources sensible in a single bedroom. The first type includes normal speaking voices or singing voices from the monitored person; the second type includes continuous breathing sounds or snores; and the third type includes continuous background noise in the environment. Therefore, the system can first classify the sound intensity (e.g. the decibel level or decibel spectrum) in the environment where the monitored person (e.g. an elderly person in a bedroom of the above-mentioned nursing home) stays. After analyzing the amplitude digital values (e.g. the decibel levels) in the above-mentioned volume data file, it is known that when the monitored person is moving or speaking in a normal manner, all of the three types of sound sources exist, and a first amplitude digital value corresponding to the combination of the three falls in a first region; when the monitored person is resting or sleeping, the second type and the third type of the sound sources exist, and a second amplitude digital value corresponding to the combination of the two falls in a second region; and when the monitored person has poor or no vital signs, the second type of sound source does not exist, and the amplitude digital value (a third amplitude digital value) falls in a third region, representing that only the background noise continues in the environment.

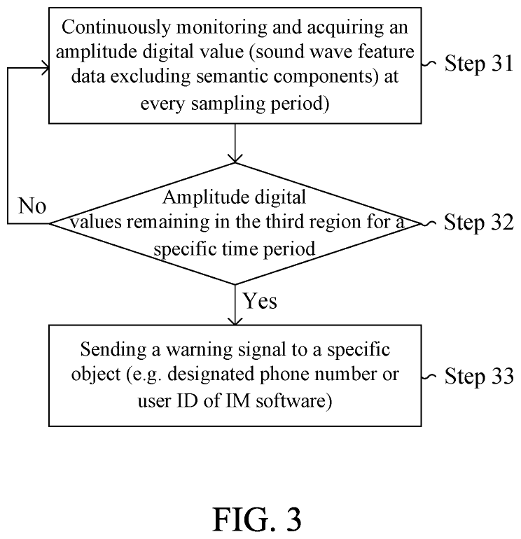

Accordingly, the feature analysis determination in the embodiment may include steps as shown in the flowchart of FIG. 3. In Step 31, the system of the present disclosure continuously monitors sounds in the bedroom to acquire an amplitude digital value (the sound wave feature data excluding semantic components) at every sampling period. Then, the amplitude digital values are sent to the determination in step 32. When the system detects that the amplitude digital values remain in the third region for a specific time period, it is determined that the monitored person has weak or no vital signs. Hence, the system automatically sends a control signal to an external system 20. In the embodiment, as shown in step 33, a warning signal is sent to a specific object (e.g. a designated phone number or a user ID of instant messaging software) to call the caregiver to give emergent treatment.

Certainly, in addition to using the amplitude digital values (e.g. the decibel level) for classification, the system could compare a segment of the sequence of time-varying amplitude digital values (e.g. the decibel spectrum) recorded in the volume data file and a preset value sequence. For example, the amplitude digital value sequence (e.g. the decibel spectrum) corresponding to the monitored person with normal breathing cycles should have a specific waveform (e.g. a first waveform). The amplitude digital value sequence corresponding to the background noise in the environment should have another specific waveform (e.g. a second waveform). When the monitored person is resting or sleeping, the second type and the third type of the sound sources exist. Therefore, the corresponding amplitude digital value sequence (e.g. the decibel spectrum) is equivalent to the combination of the first waveform and the second waveform. When the monitored person has weak or no vital signs, the first waveform does not occur so that the amplitude digital value sequence (e.g. the decibel spectrum) conforms to the second waveform. Hence, when the system detects that the amplitude digital value sequence (e.g. the decibel spectrum) changes from the combination of the first waveform and the second waveform to just the second waveform which lasts for a specific time period, it is determined that the monitored person has weak or no vital signs. Consequently, the system automatically sends a control signal to the external system 20. For example, a warning signal is sent to a specific object (e.g. a designated phone number or a user ID of instant messaging software) to call the caregiver to give emergent treatment.

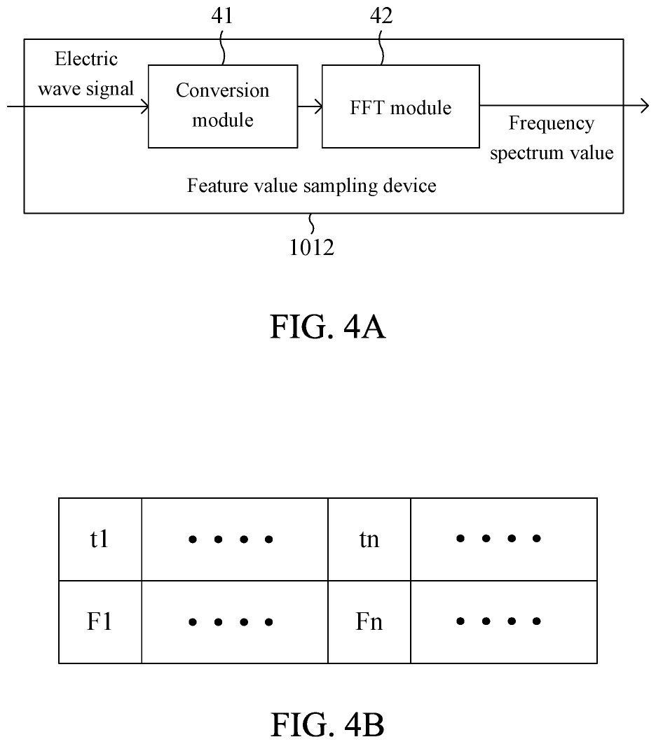

Furthermore, the feature value sampling device 1012 could be implemented by a real-time spectrum analyzer, as shown in FIG. 4A. In another embodiment, the feature value sampling device 1012 of the present disclosure includes a conversion module 41 and a fast Fourier transform module 42. The conversion module 41 uses superheterodyne technology to convert the received electric wave signal to have a lower frequency. Then, the fast Fourier transform module 42 uses fast Fourier transform (FFT) to perform the feature value sampling procedure at every sampling period. In the embodiment, the feature value is a frequency spectrum value of the electric wave signal. The feature values (i.e. intensity digital values at a plurality of frequencies) obtained at every sampling time point form an array which is then transmitted to the sound wave feature analysis module 102. The sound wave feature analysis module 102 records and temporarily stores the frequency spectrum values into the memory device 1021 as a frequency spectrum data file. The frequency spectrum data file includes, as shown in FIG. 4B, the frequency spectrum data arrays F1 . . . Fn corresponding to the sampling time points t1 . . . tn. Consequently, the frequency spectrum data file keeps the frequency spectrum feature of the sound wave, but the semantic components of the sound wave have been removed or reduced to become unrecognizable. The data processing unit 1022 of the sound wave feature analysis module 102 can make the feature analysis determination based on the frequency spectrum data file in real time.

Therefore, the frequency spectrum data array of a specific time period recorded in the frequency spectrum data file could be compared with a preset value array. For example, the frequency spectrum data array corresponding to the monitored person with normal breathing cycles should have a specific distribution (e.g. a first distribution). The frequency spectrum data array corresponding to the background noise in the environment should have another specific distribution (e.g. a second distribution). When the monitored person is resting or sleeping, the second type and the third type of the sound sources exist. Therefore, the frequency spectrum data array has a distribution equivalent to the combination of the first distribution and the second distribution. When the monitored person has weak or no vital signs, the first distribution does not occur so that the frequency spectrum data array conforms to the second distribution. Hence, when the system detects that the frequency spectrum data array changes from the combination of the first distribution and the second distribution into just the second distribution which lasts for a specific time period, it is determined that the monitored person has weak or no vital signs. Consequently, the system automatically sends a control signal to the external system 20. For example, a warning signal is sent to a specific object (e.g. a designated phone number or a user ID of instant messaging software) to call the caregiver to give emergent treatment.

Besides, the distribution state of the frequency spectrum data array can be used to distinguish different sound types representing different respiratory abnormalities such as cough, expectoration, hemoptysis, asthma or hoarseness. If the system detects that the distribution corresponding to the background noise continuing in the environment (e.g. the second distribution) combined with the sound type representing a respiratory abnormality (e.g. a third distribution) conforms to a preset frequency spectrum data array, it is determined that the monitored person may have the corresponding respiratory abnormality. Consequently, the system automatically sends a control signal to the external system 20. For example, a warning signal is sent to a specific object (e.g. a designated phone number or a user ID of instant messaging software) to call the caregiver to give particular treatment.

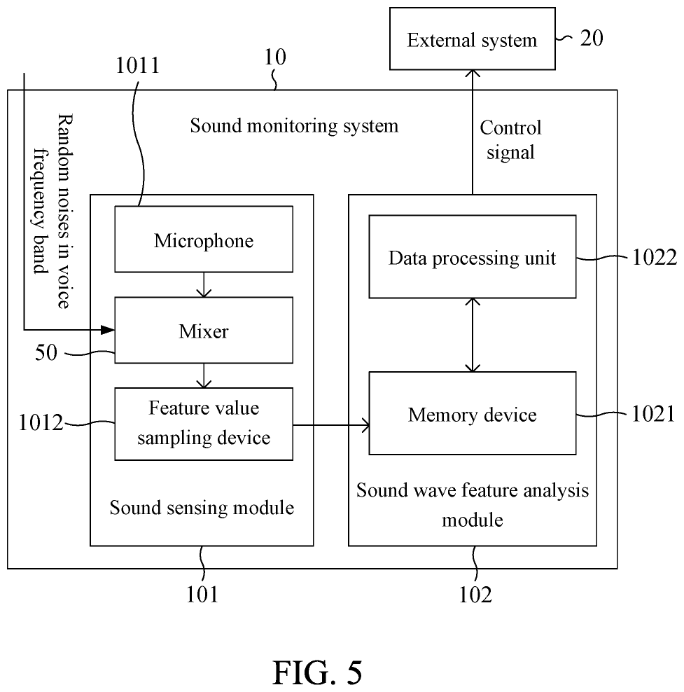

To reduce the semantic components of the sound wave to become unrecognizable, the present disclosure provides another embodiment of a sound monitoring system which is also applied in a monitoring environment, and its functional block diagram is shown in FIG. 5. It differs from the sound monitoring system 10 of FIG. 1 in that a mixer 50 is added in the sound sensing module 101. Before the electric wave signal generated by the sound sensing module 101 according to the sound wave is transmitted to the sound wave feature analysis module 102, the mixer 50 introduces random noises, lying in the voice frequency band, into the electric wave signal to remove or reduce the semantic components of the sound wave to become unrecognizable. The random noises lying in the voice frequency band have fixed energy in order to evenly affect the decibel spectrum, and will not affect the indicators, relating to the health status of the monitored person and obtained by the determination procedure. At the same time, the monitored person will not consider that his/her privacy has been violated.

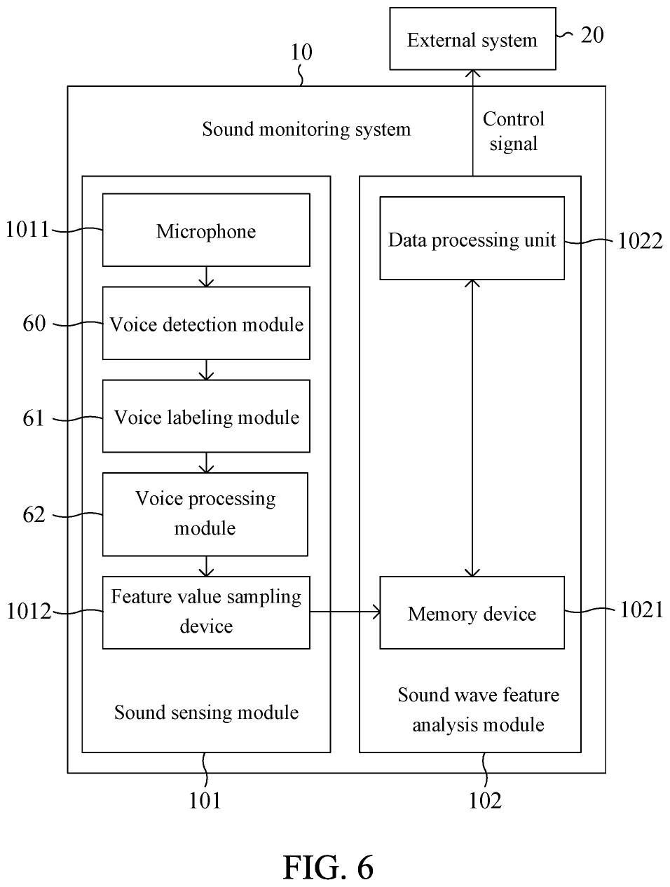

Please further refer to FIG. 6, which is a functional block diagram of a further embodiment of a sound monitoring system which is also applied in a monitoring environment according to the present disclosure. The microphone 1011 included in the sound sensing module 101 can continuously convert the sound wave (speech or other sound) generated in the monitoring environment into the electric wave signal in real time. The electric wave signal in the embodiment is sent to a voice detection module 60 to perform voice detection to detect the voice-active period. Then, a voice labeling module 61 labels the voice-active period in the electric wave signal. The labeling method could be implemented by inserting specific signs at the start point and the end point of the voice-active period, or directly mixing a masker signal over the voice-active period, in order to definitely indicate the voice-active period. The labeled electric wave signal is sent to a voice processing module 62. The voice processing module 62 generates an inverted waveform with an amplitude close to that of the waveform of the electric wave signal defined by the labeling, and then mixes the both waveforms to remove or reduce the semantic components of the sound wave to become unrecognizable. Finally, the electric wave signal with the semantic components removed is sent to the feature value sampling device 1012, and the feature value sampling device 1012 performs a feature value sampling procedure on the electric wave signal with the semantic components removed to obtain and transmit one or more feature values to the sound wave feature analysis module 102. The sound wave feature analysis module 102 records and temporarily stores the feature value(s) into the memory device 1021 as a data file (could be considered as the sound wave feature data set described above). The data file mainly includes the feature value data corresponding to the sampling time points. The data processing unit 1022 of the sound wave feature analysis module 102 can make feature analysis determination based on the data file, and further sends a control signal to an external system 20 according to the determination result. The feature value sampling device 1012 could use the embodiment of FIG. 2A or FIG. 4A, and the obtained sound wave feature data set could be an amplitude digital value sequence (e.g. the decibel spectrum) or a frequency spectrum data file. Thus, the amplitude digital value sequence (e.g. the decibel spectrum) or the frequency spectrum data file keeps the frequency spectrum feature of the sound wave, but the semantic components of the sound wave have been removed or reduced to become unrecognizable, thereby protecting the user privacy. The data processing unit 1022 of the sound wave feature analysis module 102 can make the feature analysis determination based on the amplitude digital value sequence (e.g. the decibel spectrum) or the frequency spectrum data file in real time.

While the disclosure has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A sound monitoring system applied in a monitoring environment, comprising:
a sound sensing module, receiving a sound wave generated in the monitoring environment throughout a period of time, wherein the sound wave is transformed into a sound wave feature data set through a transformation process and the transformation process removes or reduces semantic components of the sound wave to become unrecognizable; and
a sound wave feature analysis module, in communication with the sound sensing module, receiving the sound wave feature data set and making feature analysis determination according to the sound wave feature data set, and then sending a control signal according to the feature analysis determination.

2. The sound monitoring system according to claim 1, wherein the sound sensing module comprises:
a microphone continuously converting the sound wave generated in the monitoring environment into an electric wave signal in real time; and
a feature value sampling device, electrically connected to the microphone and the sound wave feature analysis module, performing a feature value sampling procedure on the electric wave signal at every sampling period to obtain one or more feature values, and transmitting the one or more feature values to the sound wave feature analysis module.

3. The sound monitoring system according to claim 2, wherein the sound wave feature analysis module comprises:
a memory device, electrically connected to the feature value sampling device, recording the feature values as a data file, the data file including feature value data corresponding to sampling time points; and
a data processing unit, electrically connected to the memory device, making the feature analysis determination based on the data file in real time, and sending the control signal to an external system according to a determination result of the feature analysis determination.

4. The sound monitoring system according to claim 3, wherein the feature value sampling device comprises a signal amplifier and an analog-to-digital converter, the signal amplifier amplifying an amplitude of the electric wave signal and the analog-to-digital converter performing the feature value sampling procedure on the amplified electric wave signal at every sampling period, wherein
the feature value is obtained by transforming the amplitude of the amplified electric wave signal into an amplitude digital value;
after obtaining one or more amplitude digital values, the one or more amplitude digital values are transmitted to the sound wave feature analysis module;
the sound wave feature analysis module records and temporarily stores the one or more amplitude digital values into the memory device as a volume data file; and
the volume data file includes amplitude digital value data corresponding to the sampling time points.

5. The sound monitoring system according to claim 4, wherein each of the amplitude digital values represents a volume at a corresponding sampling time point, and the data processing unit makes the feature analysis determination based on the volume data file by using a time-varying amplitude digital value sequence recorded in the volume data file, the feature analysis determination comprising steps of: determining that a monitored person has poor or no vital signs when the amplitude digital values falls in a preset region for a specific time period, and automatically sending the control signal to the external system,
wherein the external system is associated with a designated phone number or a user ID of instant messaging software, and the control signal is a warning signal calling a caregiver to give emergent treatment.

6. The sound monitoring system according to claim 4, wherein the data processing unit compares a time-varying amplitude digital value sequence recorded in the volume data file and a preset value sequence; determines that a monitored person has weak or no vital signs when the data processing unit detects that the amplitude digital value sequence changes from a combination of a first waveform and a second waveform to just the second waveform which lasts for a specific time period; and automatically sends the control signal to the external system,
wherein the external system is associated with a designated phone number or a user ID of instant messaging software, and the control signal is a warning signal calling a caregiver to give emergent treatment.

7. The sound monitoring system according to claim 3, wherein the feature value sampling device comprises a conversion module and a fast Fourier transform module, the conversion module receiving and converting the electric wave signal to have a lower frequency, and the fast Fourier transform module using fast Fourier transform to perform the feature value sampling procedure at every sampling period, wherein a plurality of frequency spectrum values obtained at every sampling period form an array, and the array is transmitted to the sound wave feature analysis module; the sound wave feature analysis module records and temporarily stores the frequency spectrum values into the memory device as a frequency spectrum data file; and the frequency spectrum data file includes spectrum value data arrays corresponding to the sampling time points.

8. The sound monitoring system according to claim 7, wherein the data processing unit compares the frequency spectrum data array of a specific time period recorded in the frequency spectrum data file and a preset value array; determines that a monitored person has weak or no vital signs when the data processing unit detects that the frequency spectrum data array changes from a combination of a first distribution and a second distribution into just the second distribution which lasts for a specific time period; and automatically sends the control signal to the external system,
wherein the external system is associated with a designated phone number or a user ID of instant messaging software, and the control signal is a warning signal calling a caregiver to give emergent treatment.

9. The sound monitoring system according to claim 7, wherein a distribution state of the frequency spectrum data array is used to distinguish different sound types representing different respiratory abnormalities; and
the data processing unit determines that a monitored person has one respiratory abnormality when the data processing unit detects that a distribution corresponding to a combination of a background noise and the sound type representing the one respiratory abnormality conforms to a preset frequency spectrum data array.

10. The sound monitoring system according to claim 1, wherein the sound sensing module further comprises a mixer, wherein before the electric wave signal generated by the sound sensing module according to the sound wave is transmitted to the sound wave feature analysis module, the mixer introduces random noises, lying in a voice frequency band, into the electric wave signal to remove or reduce the semantic components of the sound wave to become unrecognizable.

11. The sound monitoring system according to claim 1, wherein the sound sensing module comprises:
a microphone continuously converting the sound wave generated in the monitoring environment into an electric wave signal in real time;
a voice detection module, electrically connected to the microphone, detecting a voice-active period in the electric wave signal;
a voice labeling module, electrically connected to the voice detection module, labeling the voice-active period in the electric wave signal;
a voice processing module electrically connected to the voice labeling module, wherein after the voice processing module receives the electric wave signal with labeling, the voice processing module generates an inverted waveform with an amplitude close to a waveform of the electric wave signal defined by the labeling, mixes both waveforms to remove or reduce the semantic components of the sound wave to become unrecognizable, and sends the electric wave signal with the semantic components removed; and
a feature value sampling device, electrically connected to the voice processing module and the sound wave feature analysis module, performing a feature value sampling procedure on the electric wave signal with the semantic components removed at every sampling period to obtain one or more feature values, and transmitting the one or more feature values to the sound wave feature analysis module.

12. The sound monitoring system according to claim 11, wherein the voice-active period is labeled by inserting specific signs at a start point and an end point of the voice-active period, or directly mixing a masker signal over the voice-active period to definitely indicate the voice-active period.
